(19) United States
(12) Patent Application Publication
(10) Pub. No.: US 2009/0005709 A1
(43) Pub. Date: Jan. 1, 2009

(54) Range of motion measurement device

(76) Inventor: Raoul J. Gagne, Williamsburg, VA (US)

Correspondence Address: RAOUL J. GAGNE, 129 ROFFINGHAMS WAY, WILLIAMSBURG, VA, 23185, US

(21) Appl. No.: 11/821,821

(22) Filed: Jun. 27, 2007

(51) Int. Cl. A61B 5/103

(52) U.S. Cl. 600/594; 600/587; 600/595

(57) ABSTRACT

Apparatus for simultaneously measuring the range of motion in a full 360 degree by 180 degree dual axis range. The devices and software applications allow for real time measurement of total spine and extremity movements by analyzing both pitch and roll angles simultaneously. This provides the tester with an extremely accurate account of range of motion without the usual inaccuracies of individual turning into or away from the set movement plane. Furthermore, the small and easy positioning device and application software allow the system to function without dependency on gravity for accuracy of motion measurement.

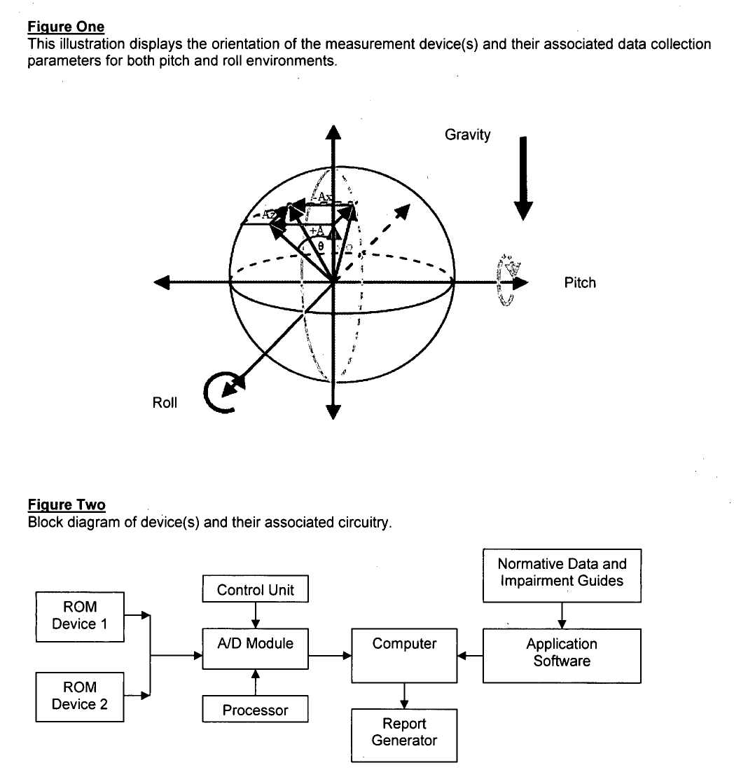

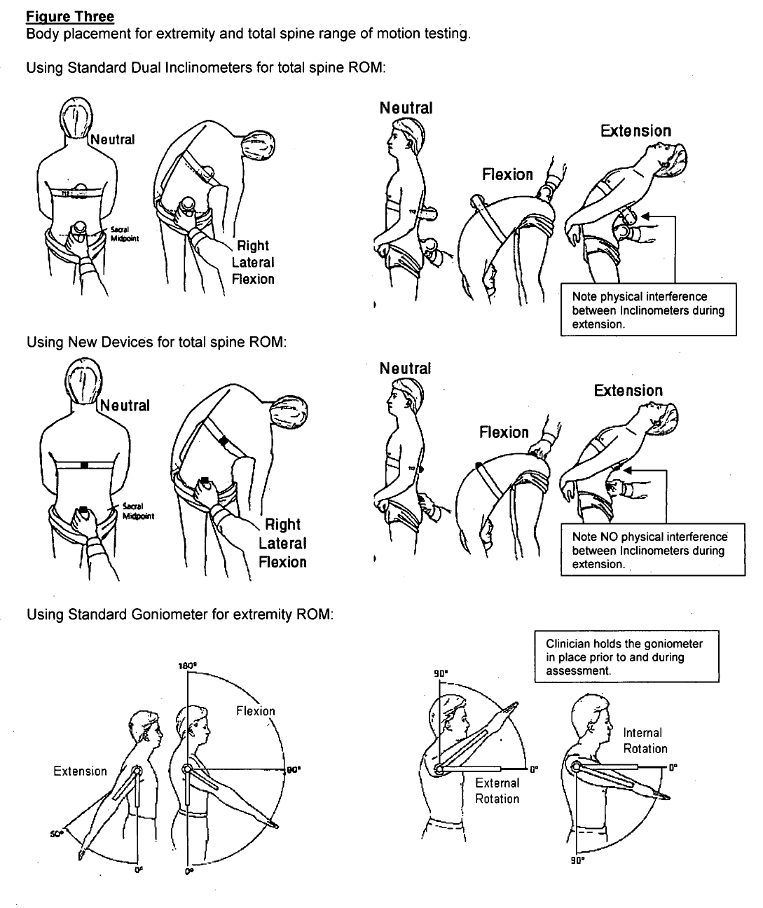

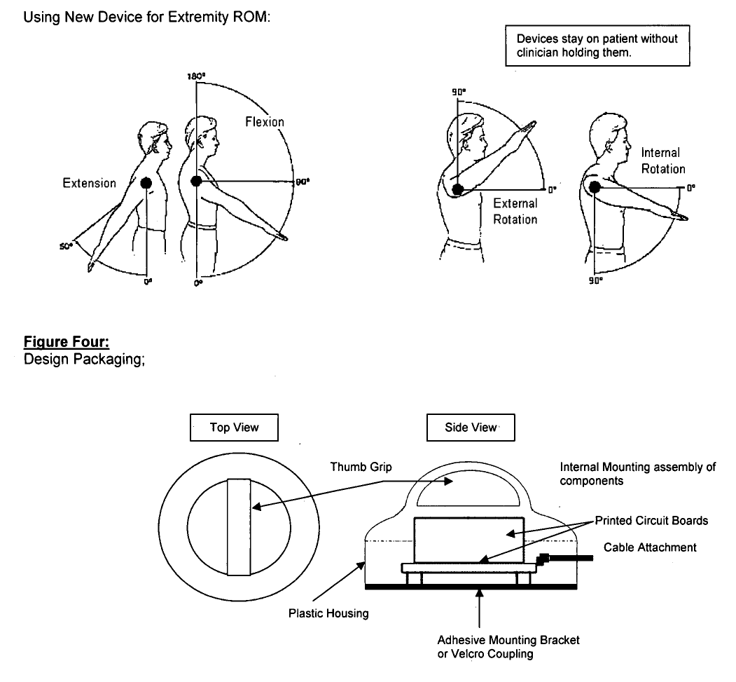

### BACKGROUND OF THE INVENTION

Historically all human range of motion in a clinical setting was diagnosed through the use of a goniometer or a single or dual set of inclinometers. These devices are either manual in nature or they are linked to a computerized system for measurement and subsequent reporting of results. The goniometer is the most common method of testing; however, is the most unreliable due to human error in body positioning. The goniometer is used primarily for extremities. The new device only requires a single component strapped to the extremity to measure range of motion and eliminates inaccurate setup. The inclinometer methodologies are used for the total spine. It is extremely difficult to calculate accurate readings of spinal movement with a single inclinometer. The inclinometer methods in a dual mode are the most used and provide the ability to segment out the portion of the spine showing movement. However, the limitations of the dual inclinometers are that they are gravity based and can only measure in one plane. This forces the evaluee to lay in a supine position when testing for cervical range of motion and secondarily does not compensate for errors in individuals leaning into or away form the testing plane (i.e. straight up and down) To change from flexion and extension to lateral movements requires a new set up of the inclinometers. They are required to be aligned with the direction of movement in order to measure properly. The new device works with a series of accelerometers for high reliability and accuracy providing no dependency on gravity and no requirement for change of positioning when moving from flexion and extension to lateral movements. Size of the inclinometers has also presented a hardship in the attachment to the tested individual. They are all heavy and large and require strapping to hold them in position. The new device is less than 1/10^{th} the weight of present inclinometers and is ¼ the size, and can be mounted via adhesive electrode or Velcro allowing ease of use in set up and increased accuracy due to non-slippage during body movements.

### CROSS REFERENCE AND/OR RELATED APPLICATIONS

| | | |
|---|---|---|
| 5188121 | February 1993 | Hanson |
| 5163228 | November 1992 | Edwards, et al. |
| 6792801 | September 2004 | Hoggan, et al. |
| 5373858 | December 1994 | Rose, et al. |
| 5758658 | June 1998 | Petragallo |
| 5588444 | December 1996 | Petragallo |
| 6792801 | September 2004 | Hoggan. et al. |

### OTHER REFERENCES

Range of Motion References:
Guides to the Evaluation of Permanent Impairment American Medical Association, 4^{th} ed., pp. 112-135 (1993).
Guides to the Evaluation of Permanent Impairment American Medical Association, 3^{rd} ed., pp. 81-102 (1990).
Guides to the Evaluation of Permanent Impairment American Medical Association, 4^{th} ed., pp. 90-92 (1993).
Guides to the Evaluation of Permanent Impairment American Medical Association, 3^{rd} ed., pp. 20-38, 101 (1990).

### BRIEF SUMMARY OF THE INVENTION

I, Raoul Gagne, have invented a new design for measuring range of motion, as set forth in the following specification. The device is used by a clinician to measure an individual's total spine or extremity range of motion.

### FIGURE DESCRIPTION

Figure One

This illustration displays the orientation of the measurement device and its associated data collection parameters for both pitch and roll environments.

Figure Two

Block diagram of device(s) and their associated circuitry.

Figure Three

Samples of body placement for extremity and total spine range of motion testing.

Figure Four

Design packaging.

### DETAILED DESCRIPTION OF THE INVENTION

The range of motion device is the smallest and lowest power fully calibrated module in the industry. They combine advanced 2 and 3-axis measurement algorithms with the reliability and performance of silicon accelerometers. Similar systems are deployed throughout the world in diverse applications such as pipeline inspection, vehicle leveling, ROV navigation and satellite positioning. The devices are surface-mount tilt and vibration sensors that provide simple, reliable solutions for on/off tilt angle switching and omnidirectional movement sensing. These types of sensors have become the industry standard for GPS and RFID tracking applications, where they provide intelligent power management to interrupt and “wake-up” a microcontroller when activity is sensed. They are designed to be extremely low power, consuming virtually no power when at rest and generating a digital output when moved. They are fully passive, require no signal conditioning, and can be used in a triggering circuit that draws as little as 0.25 uA of continuous current.

The devices are extremely small, close to the footprint of a quarter, with outputs in both an analog and digital environment. Communication is through a standard serial cable or custom cable link to a microprocessor. Software deciphers the output and displays the corresponding measurement in degrees for the movement pattern. Furthermore the software has the capability to cross compare impairment guidelines and coefficient of variation between multiple trials.

The device can be used by itself for extremity range of motion or in combination with a secondary identical device to segment out portions of the spine to calculate range of motion. The device has the capability to continually monitor range of motion parameters over an extended timeframe if required for clinical or industrial applications.

The device is manufactured as a two part printed circuit board in a plastic package with a thumb grip on top to allow for body positioning. The unit is sealed and has a Velcro bottom to marry to Velcro strapping material during body positioning.

1. I, Raoul Gagne, claim that this new device will be the first to provide accurate range of motion measurements for both extremity and total spine in both x and y axis's simultaneously.

2. Further to claim (I), I claim that this device provides increased ease of use to the clinician through less patient set up and increasing accuracy due to single step body positioning of the device(s) for both flexion and extension and lateral left and right movements.

3. Further to claim (I) and (II), this device is smaller and easier to place on the body than any other previously marketed device for the application of range of motion testing thus providing less time in setup due to no required strapping and eliminating interference between old devices such as inclinometers during back extension measurements.
